(19) United States
(12) Patent Application Publication
(10) Pub. No.: US 2025/0012812 A1
(43) Pub. Date: Jan. 9, 2025

(54) Biomarker for Detecting Hepatocytes With Bile Ductular Proliferation

(71) Applicants: The University of Tokyo, Tokyo (JP); Kanagawa Prefectural Hospital Organization, Kanagawa (JP); Tokyo Institute of Technology, Tokyo (JP); National University Corporation Kanazawa University, Ishikawa (JP)

(72) Inventors: Motoharu Seiki, Tokyo (JP); Taro Yamashita, Tokyo (JP); Hikari Okada, Ishikawa (JP); Shuichi Kaneko, Ishikawa (JP); Naohiko Koshikawa, Kanagawa (JP); Nobuaki Funahashi, Tokyo (JP)

(21) Appl. No.: 18/291,509

(22) PCT Filed: Jul. 22, 2022
(86) PCT No.: PCT/JP2022/028466

§ 371 Date: Jan. 23, 2024

(30) Foreign Application Priority Data
Jul. 26, 2021 (JP) 2021-121616

(51) Int. Cl. G01N 33/68

(52) U.S. Cl. CPC G01N 33/6893; G01N 2800/085

(57) ABSTRACT

The present invention provides a method of detecting a biomarker for detecting hepatocytes with bile ductular proliferation, the method including: detecting laminin γ2 single chain or a nucleic acid encoding the same as the biomarker in a specimen.

### TECHNICAL FIELD

The present invention broadly provides a biomarker for detecting hepatocytes with bile ductular proliferation, and various applications using the same.

### BACKGROUND ART

Laminins are a group of heterotrimeric proteins found in the basal lamina and form a part of the basement membrane. These proteins are classified based on three non-identical polypeptides that complex with each other to form laminin structures. These three polypeptides are identified as alpha (α), beta (β), and gamma (γ) chains, each with several molecular species (for example, α1-α5, β1-β3,and γ1-γ2). Laminin 332 (also referred to as laminin 5 or LN5) is present in the basal lamina and is known to be abundant in the basement membrane located between epithelial cells and the connective tissue that lines the epithelial cells. The structure of laminin 332 is unique among known laminins in that laminin 332 is the only laminin with a structure containing a gamma-2 (γ2) chain that forms laminin 332 when complexed with α3 and β3 chains. Physiologically, laminin 332 is produced by epithelial cells and is known to be able to promote cell adhesion, proliferation, differentiation and/or migration. For example, when laminin 332 is secreted from epithelial cells, it is susceptible to protease degradation (for example, by membrane type 1-matrix metalloproteinase-1 (MT1-MMP)). In some cases, laminin 332 is processed toward the N-terminus of the gamma-2 chain sequence to produce fragments with epidermal growth factor (EGF)-like activities, including promotion of cell migration and invasion (Koshikawa et al., J. Cell Biol., (2000) 148: pp. 615-624).

It is known that increased concentrations or levels of laminin γ2 single chain in biological samples such as blood are associated with cancer, such as colorectal cancer and/or bladder cancer. For example, WO 2014/027701 discloses a method of providing a diagnosis, prognosis or risk classification to a subject having cancer or at risk of having cancer, the method including: a step of comparing the laminin γ2 single chain concentration in a sample from the subject with a reference laminin γ2 single chain concentration value, in which a laminin γ2 single chain concentration in the sample higher than the reference laminin γ2 single chain concentration value identifies a subject as having cancer or at increased risk of developing cancer. In addition, Patent Publication JP-A-2011-209281 discloses a method and kit for testing for urological cancer, which are characterized by measuring laminin γ2 single chain in urine collected from a subject.

### CITATION LIST

### Patent Documents

Patent Document 1: WO 2014/027701

Patent Document 2: Patent Publication JP-A-2011-209281

Patent Document 3: WO 2017/057778

### Non-Patent Document

Non-Patent Document 1: Koshikawa et al., J. Cell Biol., (2000) 148: pp. 615-624

### SUMMARY

### Technical Problem

An object of the present invention is to provide an application of laminin γ2 single chain as a novel biomarker.

### Solution to Problem

In various liver diseases and disorders, an abnormal increase in bile duct structures around Glisson's capsule occurs. This phenomenon is called bile ductular reaction because it is a proliferation of bile ducts similar to bile ductules, which are thin luminal structures connecting interlobular bile ducts and hepatocytes.

The present inventors have found that laminin γ2 single chain is found in hepatocytes with bile ductular proliferation (bile ductular reaction), thus leading to realization of the present invention.

That is, the present application includes the following inventions.

[1] A method of detecting a biomarker for detecting hepatocytes with bile ductular proliferation which includes the step of: detecting laminin γ2 single chain or a nucleic acid encoding the same as the biomarker in a specimen.

[2] The method according to [1], in which hepatocytes with bile ductular proliferation are cells having a liver disease causing bile ductular proliferation.

[3] The method according to [2], in which the liver disease causing bile ductular proliferation is selected from the group consisting of primary biliary cholangitis, acute hepatitis, fulminant hepatitis, liver failure, primary sclerosing cholangitis, hepatic fibrosis, biliary atresia, and cholestasis.

[4] The method according to any one of [1] to [3], in which, in a case where laminin γ2 single chain or a nucleic acid encoding the same is detected, the specimen is likely to contain hepatocytes with bile ductular proliferation or hepatocytes having a liver disease causing bile ductular proliferation.

[5] The method according to any one of [1] to [4] which further includes the step of: detecting a bile ductular proliferation-related marker other than laminin γ2 single chain or a nucleic acid encoding the same.

[6] The method according to [5], in which the bile ductular proliferation-related marker is one or a plurality of makers selected from the group consisting of SOX9 AQP1, CK7, EpCAM, and Dlk1.

[7] The method according to any one of [1] to [6], in which the detection of laminin γ2 single chain or a nucleic acid encoding the same is performed over time.

[8] The method according to [7], in which change in the amount of laminin γ2 single chain in a specimen or a nucleic acid encoding the same over time is compared with change in the amount of CK19 in the specimen or a nucleic acid encoding the same over time.

[9] The method according to any one of [1] to [8], in which, the specimen is derived from a subject suspected of having hepatocytes with bile ductular proliferation or hepatocytes having a liver disease causing bile ductular proliferation.

[10] The method according to [9], in which the specimen is derived from blood or urine of the subject.

[11] A method of differentiating hepatocytes with bile ductular proliferation from hepatocytes without bile ductular proliferation which includes the step of: detecting laminin γ2 single chain or a nucleic acid encoding the same in a specimen, in which in a case where laminin γ2 single chain or a nucleic acid encoding the same is detected, the specimen is determined to contain hepatocytes with bile ductular proliferation, and in a case where laminin γ2 single chain or a nucleic acid encoding the same is not detected, the specimen is determined not to contain hepatocytes with bile ductular proliferation.

[12] The method according to [11], in which hepatocytes with bile ductular proliferation are cells having a liver disease causing bile ductular proliferation.

[13] The method according to [12], in which the liver disease causing bile ductular proliferation is selected from the group consisting of primary biliary cholangitis, acute hepatitis, fulminant hepatitis, liver failure, primary sclerosing cholangitis, hepatic fibrosis, biliary atresia, and cholestasis.

[14] A biomarker for detecting hepatocytes with bile ductular proliferation, containing: laminin γ2 single chain or a nucleic acid encoding the same.

[15] The biomarker according to [14], in which hepatocytes with bile ductular proliferation are cells having a liver disease causing bile ductular proliferation.

[16] A kit for detecting hepatocytes with bile ductular proliferation, including: a reagent for detecting laminin γ2 single chain or a nucleic acid encoding the same.

[17] The kit according to [16], in which hepatocytes with bile ductular proliferation are cells having a liver disease causing bile ductular proliferation.

[18] The kit according to [17], in which the liver disease causing bile ductular proliferation is selected from the group consisting of primary biliary cholangitis, acute hepatitis, fulminant hepatitis, liver failure, primary sclerosing cholangitis, hepatic fibrosis, biliary atresia, and cholestasis.

[19] The kit according to any one of to [18], further including: a reagent for detecting CK19 or a nucleic acid encoding the same.

### Advantageous Effects of Invention

According to the present invention, it is possible to diagnose or differentiate a liver disease causing bile ductular proliferation using detection of laminin γ2 single chain, which is a novel biomarker relating to bile ductular proliferation, as an indicator.

Cytokeratin 19 (CK19) is known as a marker for bile ductules and is characterized in that it is constantly expressed in bile duct epithelial cells. On the other hand, laminin γ2 single chain is characterized in that its expression disappears with maturation of bile ducts and is promoted again through bile duct proliferation (appearance of immature bile ducts). For this reason, by monitoring laminin γ2 single chain, change in hepatocytes over time, such as a state of formation of bile ducts, can also be confirmed.

In addition, WO 2017/057778 discloses laminin γ2 single chain as a biomarker not only for liver cancer but also for liver cirrhosis. Liver cirrhosis differs from liver fibrosis in that liver fibrosis, a liver disease causing bile ductular proliferation, is a state where no nodules are formed, while liver cirrhosis is a state where nodules are completely formed in a lesion part. In this manner, the disease targeted by the biomarker disclosed in WO 2017/057778 is not a liver disease causing bile ductular proliferation.

### BRIEF DESCRIPTION OF DRAWINGS

FIG. 1 shows results of immunohistological staining to verify immunoreactivity of each laminin chain antibody to mouse laminin chains and shows localization of Ln-γ2, Ln-β3, and Ln-α3 expression in mouse skin.

FIG. 2 shows results of immunostaining and hematoxylin-eosin (HE) staining using NASH mouse model livers in which non-alcoholic steatohepatitis (NASH) is induced through an ultra-high fat choline-deficient methionine-reduced diet (CDAA). The upper panel of FIG. 2A shows the staining results of mouse liver after 2 weeks of CDAA feeding, and the lower panel shows the staining results of mouse liver after 4 weeks of CDAA feeding.

The upper panel of FIG. 2B shows the staining results of mouse liver after 8 weeks of CDAA feeding, and the lower panel shows the staining results of mouse liver after 10 weeks of CDAA feeding.

FIG. 3 shows results of immunostaining and HE staining using normal diet (ND)-fed mouse livers (control livers).

FIG. 4 shows results of Azan staining using CDAA diet-induced NASH mouse model livers.

FIG. 5 shows results of immunostaining and HE staining using atherogenic plus high-fat (Ath+HF) diet-induced NASH mouse model livers. FIG. 5A shows results after 17 weeks of Ath+HF feeding.

FIG. 5B shows results after 30 weeks of Ath+HF feeding.

FIG. 5C shows results after 60 weeks of Ath+HF feeding.

FIG. 6 shows results of Azan staining using Ath+HF diet-induced NASH mouse model livers.

### DESCRIPTION OF EMBODIMENTS

Hereinafter, embodiments of the present invention (hereinafter referred to as the “present embodiments”) will be described, but the scope of the present invention should not be limitedly interpreted by the embodiments below.

### (Detection Method)

According to a first aspect, there is provided a method of detecting a biomarker for detecting hepatocytes with bile ductular proliferation, the method including a step of: detecting laminin γ2 single chain or a nucleic acid encoding the same as the biomarker in a specimen.

Bile ductules, the most peripheral branches of the intrahepatic bile duct system, connect hepatocytes at the margins of hepatic lobules to interlobular bile ducts within the portal vein area. These bile ductules are highly reactive tissue components, and the phenomenon that the number of bile ductules increase in many hepatobiliary diseases is called bile ductular proliferation.

Bile ductular proliferation is a lesion recognized in various liver diseases, and examples of such liver diseases include primary biliary cholangitis, acute hepatitis, fulminant hepatitis, liver failure, primary sclerosing cholangitis, liver fibrosis, biliary atresia, cholestasis, liver disorders due to lifestyle-related diseases (alcoholic liver disease and non-alcoholic fatty liver disease), and drug-induced liver disease.

Laminin γ2 single chain (in the present specification, also referred to as “Ln-γ2m”) refers to a γ2 chain, which is a constituent element of laminin 332, expressed as a monomer. Laminin 5 is one of important components of the basement membrane, and is a heterotrimer in which three polypeptides, α3, β, and γ2 chains, associate at a coiled-coil structural part. Among the three polypeptide chains, the γ2 chain has been reported to be expressed as a monomer in malignant cancer cells. In the present specification, to distinguish the γ2 chain expressed as a trimer from the “laminin 332 γ2 chain”, the γ2 chain expressed as a monomer is referred to as “laminin γ2 single chain” or “laminin γ2 chain”.

Laminin γ2 single chain can be derived from any organism and can include amino acid sequences derived from higher eukaryotes including mammals.

Although not intended to be limiting, human laminin γ2 single chain detected as a biomarker may be as follows:
1) a protein consisting of the amino acid sequence shown in SEQ ID No: 1;
2) a protein which consists of an amino acid sequence in which one or several amino acids, preferably one, two, three, four, five, six, seven, eight, nine or ten amino acids per 100 amino acids in the amino acid sequence of SEQ ID NO: 1 are deleted, substituted or added and retains the function of laminin γ2 single chain; and
3) a protein which consists of an amino acid sequence having at least 80%, preferably 90% or more, 91% or more, 92% or more, 93% or more, 94% or more, 95% or more, 96% or more, 97% or more, 98% or more, or 99% or more sequence identity to SEQ ID NO: 1 and retains the function of laminin γ2 single chain.

As used herein, the “amino acid sequence in which one or several amino acids are deleted, substituted and/or added” means a mutant amino acid sequence in which a number of amino acids are deleted, substituted and/or added to the extent that the desired function is not lost when compared to the amino acid sequence identified by a sequence number. Amino acids may refer to natural and synthetic amino acids, as well as amino acid analogs and amino acid mimetics that function similarly to natural amino acids. Amino acids may be either L-amino acids or D-amino acids. Natural amino acids are amino acids encoded by the genetic code and amino acids modified post-translationally within cells.

Substitution is preferably conservative amino acid substitution. This is because conservative amino acid substitution is likely to result in substantially the same structure or properties as a protein identified by a sequence number.

Laminin γ2 single chain may be in any form as long as its presence can be detected, and a nucleic acid encoding laminin γ2 single chain may be detected. As used herein, “nucleic acid” means not only DNA but also RNA such as mRNA or salts thereof.

Although not intended to be limiting, a nucleic acid encoding laminin γ2 single chain may be as follows:
1) a nucleic acid consisting of a base sequence of SEQ ID NO: 2;
2) a nucleic acid that can hybridize with a nucleic acid consisting of a base sequence complementary to the base sequence of SEQ ID NO: 2 under stringent conditions, preferably under highly stringent conditions, in which a protein encoded thereby retains the function of laminin γ2 single chain;
3) a nucleic acid which consists of a base sequence in which one or several bases, preferably one, two, three, four, five, six, seven, eight, nine or ten bases per 100 bases in the base sequence of SEQ ID NO: 2 are deleted, substituted or added and in which a protein encoded thereby retains the function of laminin γ2 single chain; and
4) a nucleic acid which consists of a base sequence having at least 80%, preferably 90% or more, 91% or more, 92% or more, 93% or more, 94% or more, 95% or more, 96% or more, 97% or more, 98% or more, or 99% or more sequence identity to SEQ ID NO: 2 and in which a protein encoded thereby retains the function of laminin γ2 single chain.

Here, the stringent conditions refer to conditions under which specific hybrids are formed and non-specific hybrids are not formed. The stringent conditions are easily determined by those skilled in the art and are generally empirical experimental conditions depending on the base length of nucleic acids, the washing temperature, and the salt concentration of a buffer solution. In general, longer bases require higher temperatures for proper annealing, and shorter bases require lower temperatures. Hybridization generally relies on the ability of complementary strands to re-anneal in an environment slightly below their melting points.

As used herein, the expression “highly stringent conditions” refers to conditions designed to allow hybridization of DNA strands that have a high degree of complementarity in their nucleic acid sequences and exclude hybridization of DNA with a significant number of mismatches. Stringency can vary, for example, due to the concentration of sodium chloride and sodium citrate (SSC). For example, the manual for the ECL direct nucleic acid labeling and detection system (manufactured by Amersham Pharmacia Biotech, Inc.) states that 0.1×SSC constituting a primary wash buffer can be replaced with 0.50×SSC to achieve highly stringent conditions.

Laminin γ2 single chain or a nucleic acid encoding the same can be detected through a usual method. For example, a method of measuring laminin γ2 single chain as a protein can be performed using any method of detecting and measuring the presence of proteins contained in a specimen, and examples thereof include immunoassay, an aggregation method, turbidimetry, a western blotting method, and a surface plasmon resonance (SPR) method.

An immunoassay uses a detectably labeled anti-laminin γ2 single chain antibody or an antibody (secondary antibody) against a detectably labeled anti-laminin γ2 single chain antibody. Depending on the antibody labeling method, it is classified into an enzyme immunoassay (EIA or ELISA), a radioimmunoassay (RIA), a fluorescence immunoassay (FIA), a fluorescence polarization immunoassay (FPIA), a chemiluminescence immunoassay (CLIA), and the like, and all of these can be used in the method of the present invention.

Antibodies labeled with enzymes such as peroxidases and alkaline phosphatases are used in the ELISA method, antibodies labeled with radioactive substances such as 125I, 131I, 35S, and 3H are used in the RIA method, antibodies labeled with fluorescent substances such as fluorescein isothiocyanate, rhodamine, dansyl chloride, phycoerythrin, tetramethylrhodamine isothiocyanate, and a near-infrared fluorescent material are used in the FPIA method, and antibodies labeled with luminescent substances such as a luciferase, luciferin, and aequorin are used in the CLIA method. In addition, antibodies labeled with nanoparticles such as gold colloids and quantum dots can also be detected.

In addition, in the immunoassay, detection can be performed by labeling an anti-laminin γ2 single chain antibody with biotin and bonding it to avidin or streptavidin labeled with an enzyme or the like.

Among the immunoassays, the ELISA method using an enzyme label is preferable because it allows measurement of antigens simply and quickly.

The ELISA method includes a competitive method and a sandwich method. In the competitive method, an anti-laminin γ2 single chain antibody is immobilized on a solid-phase carrier such as a microplate, a specimen and enzyme-labeled laminin γ2 single chain are added thereto to cause an antigen-antibody reaction. Once washed, the reactant is reacted with an enzyme substrate to develop color, and the absorbance is measured. The more laminin γ2 single chain in a specimen, the weaker the color, and the less laminin γ2 single chain in the specimen, the stronger the color. Therefore, the amount of laminin γ2 single chain can be determined using a calibration curve.

In the sandwich method, an anti-laminin γ2 single chain antibody is immobilized on a solid-phase carrier, and a specimen is added thereto to cause a reaction. Then, an enzyme-labeled anti-laminin γ2 single chain antibody that recognizes another epitope is added thereto to cause a reaction. After washing, the reactant can be reacted with an enzyme substrate to develop color, and the absorbance can be measured to obtain the amount of laminin γ2 single chain. In the sandwich method, after an antibody immobilized on a solid-phase carrier is reacted with laminin γ2 single chain in a specimen, a non-labeled antibody (primary antibody) may be added thereto and an antibody (secondary antibody) against this non-labeled antibody may be further added thereto.

In a case where an enzyme is a peroxidase, 3,3′-diaminobenzidine (DAB), 3,3′,5,5′-tetramethylbenzidine (TMB), o-phenylenediamine (OPD), or the like can be used as an enzyme substrate. In a case where an enzyme is an alkaline phosphatase, p-nitropheny phosphate (NPP) or the like can be used.

In the present specification, “solid-phase carrier” is not particularly limited as long as it is a carrier on which an antibody can be immobilized, and examples thereof include microtiter plates made of glass, metal, a resin, and the like, substrates, beads, nitrocellulose membrane, nylon membranes, and PVDF membranes, and targets can be immobilized on these solid-phase carriers through a well-known method.

In addition, among the above-described immunoassays, an aggregation method is also preferable as a method that can simply detect a trace amount of protein. Examples of aggregation methods include a latex agglutination method in which latex particles are bound to antibodies.

When anti-laminin γ2 single chain antibodies are bound to latex particles and mixed with a specimen, the antibody-bound latex particles are aggregated in the presence of laminin γ2 single chain. Then, the antigen concentration can be determined by irradiating a sample with near-infrared light and quantitatively determining aggregates through absorbance measurement (turbidimetry) or scattered light measurement (nephelometry).

In the above-described methods, an antibody that recognizes laminin 5 γ2 chain, that is, a γ2 chain constituting a trimer, may be used as an anti-laminin γ2 single chain antibody. In addition, in the immunoassays, antibodies that recognize laminin γ2 single chain fragments processed by MMP or the like may also be used.

Both monoclonal and polyclonal anti-laminin γ2 single chain antibodies can be produced according to well-known methods. Monoclonal antibodies can be obtained by, for example, isolating antibody-producing cells from a non-human mammal immunized with laminin γ2 single chain or fragments thereof, fusing the isolated cells with myeloma cells to produce hybridomas, and purifying antibodies produced by the hybridomas. In addition, polyclonal antibodies can be obtained from sera of animals immunized with laminin γ2 single chain or fragments thereof.

Existing antibodies may be used as anti-laminin γ2 single chain antibodies. For example, examples of antibodies that specifically recognize laminin γ2 single chain without recognizing laminin 5 γ2 chain include 1H3 monoclonal antibodies (Sarosdy, M. F. et al., The Journal of Urology, 154:379 to 384, 1995). In addition, examples of antibodies that recognize laminin 5 γ2 chain and laminin γ2 single chain include D4B5 monoclonal antibodies (Sarosdy, M. F. et al., The Journal of Urology, 154:379 to 384, 1995) and 2778 polyclonal antibodies (Koshikawa et al., Cancer Res. 68:530, 2008).

A method of detecting laminin γ2 single chain is not limited to a method of detecting the presence of laminin γ2 single chain itself as a protein, and expression of a gene encoding laminin γ2 single chain may be confirmed. Examples of genes include not only DNA but also RNA such as microRNA, siRNA, tRNA, snRNA, mRNA, and non-coding RNA. For example, the transcription level of laminin γ2 single chain can be measured through an RT-PCR method or a Northern hybridization method in which mRNA encoding laminin γ2 single chain is extracted according to a standard method and this mRNA is used as a template. Non-coding RNA can be detected using, for example, a next generation sequencer.

As used herein, “specimen” broadly means a biological material containing laminin γ2 single chain or a nucleic acid encoding the same, preferably a biological material derived from a subject who is suspected of having hepatocytes with bile ductular proliferation or hepatocytes having a liver disease causing bile ductular proliferation at present or in the future. Any cells, tissues, or body fluids can be used to obtain a specimen. Such cells, tissues, and body fluids may include tissue sections such as biopsy or autopsy samples, frozen sections collected for histological purposes, blood (such as whole blood), plasma, serum, sputum, stool, tears, mucus, saliva, bronchoalveolar lavage (BAL) fluids, hair, skin, red blood cells, platelets, interstitial fluid, ocular lens fluid, cerebrospinal fluid, sweat, nasal mucus, synovial fluid, leucorrhea, amniotic fluid, semen, and the like. Cells and tissues may include lymphatic fluid, ascites, gynecological fluid, urine, peritoneal fluid, cerebrospinal fluid, and the like. Among these, blood, serum, and plasma are preferable because they can be collected with minimal invasiveness and are specimens used in many common tests.

As used herein, “subject” is not particularly limited as long as it is an animal in which laminin γ2 single chain is expressed in hepatocytes, and refers to any vertebrate animals including, for example, human or non-human primates (for example, monkeys such as cynomolgus monkeys, rhesus macaque, and chimpanzees) and other mammals (for example, cows, pigs, camels, llamas, horses, rabbits, sheep, hamsters, guinea pigs, cats, dogs, rats, and mice). Depending on embodiments, a subject may be a human or a non-human animal. Although not intended to be limiting, a subject is assumed to be a subject who is suspected of having hepatocytes with bile ductular proliferation or hepatocytes having a liver disease causing bile ductular proliferation at present or in the future.

In a case where the presence of laminin γ2 single chain or a nucleic acid encoding the same is detected in a specimen, it can be determined that there is a possibility or a high possibility that the specimen may contain hepatocytes with bile ductular proliferation or hepatocytes having a liver disease causing bile ductular proliferation. For this reason, confirmation of the presence of laminin γ2 single chain or a nucleic acid encoding the same in a specimen can be used to diagnose a liver disease causing bile ductular proliferation or to assist in the diagnosis.

The number of times of performing detection of laminin γ2 single chain or a nucleic acid encoding the same is not particularly limited. Change in lesions over time can also be determined by periodically collecting specimens from the same subject and comparing the expression level of laminin γ2 single chain or a nucleic acid encoding the same from one another. The presence of the existing marker CK19 may be detected in parallel with the detection of laminin γ2 single chain or a nucleic acid encoding the same.

The accuracy of diagnosis can be improved by detecting well-known biomarkers of a liver disease causing bile ductular proliferation in addition to laminin γ2 single chain or a nucleic acid encoding the same. Examples of such markers include bile ductular proliferation-related markers such as Sry-related HMG box transcription factor 9 (SOX9), aquaporin 1 (AQP1), cytokeratin 7 (CK7), epithelial cell adhesion molecule (EpCAM), and Delta-like 1 homolog (Dlk1). These genes are constantly expressed in bile duct epithelial cells, and their expression can be detected in the proliferated bile ductules, and therefore, they can serve as bile ductular proliferation-related markers.

A medical doctor may perform diagnosis of a liver disease causing bile ductular proliferation in parallel with detection of biomarkers. Determination of a liver disease causing bile ductular proliferation can also be performed with the assistance of clinical laboratory technicians or medical devices.

For subjects diagnosed with a liver disease causing bile ductular proliferation, treatment methods known to those skilled in the art for each disease can be applied. Although not intended to be limiting, the following are known methods of treating a liver disease causing bile ductular proliferation:
primary biliary cholangitis: administration of ursodeoxycholic acid or bezafibrate;
acute hepatitis: administration of antiviral drugs for viral cases, immunosuppressants such as steroids or azathioprine for autoimmune cases, and administration of ursodeoxycholic acid for drug-induced cases if cholestasis persists while checking the progress after discontinuing the drug;
fulminant hepatitis, liver failure, and primary sclerosing cholangitis: liver transplantation;
hepatic fibrosis: administration of branched-chain amino acid preparations for hypoalbuminemia, lactulose or rifaximin for hyperammonemia, and beta blockers or proton-pump inhibitors for portal hypertension;
biliary atresia: biliary reconstruction surgery; and cholestasis: administration of ursodeoxycholic acid or administration of nalfurafine hydrochloride for skin pruritus.

For patients diagnosed with a liver disease causing bile ductular proliferation, pharmaceutical compositions containing active ingredients for treating diseases can be administered.

The administration routes of the compositions are not particularly limited, and the compositions can be administered orally or parenterally. Examples of compositions suitable for oral administration include granules, fine granules, powders, hard capsules, soft capsules, syrups, emulsions, suspensions, or solutions. Examples of compositions suitable for parenteral administration include injections for intravenous, intramuscular, or subcutaneous administration, drops, suppositories, transdermal absorption agents, transmucosal absorption agents, nasal drops, ear drops, eye drops, and inhalants. It is also intended that preparations prepared as pharmaceutical compositions in dry powder forms, such as freeze-dried products, be dissolved when in use and used as injections or drops.

The compositions may include solid or liquid preparation additives. The preparation additives may be either organic or inorganic substances. In a case of producing oral solid preparations, for example, an excipient is added to a substance selected from the group consisting of the above-described compounds as active ingredients or salts thereof, hydrates thereof, and solvates thereof, a binder, a disintegrant, a lubricant, a coloring agent, a flavoring agent, and the like are further added thereto as necessary, and then preparations in the forms of tablets, coated tablets, granules, powders, capsules, and the like can be prepared through a usual method.

Examples of excipients include lactose, sucrose, white sugar, glucose, corn starch, starch, talc, sorbitol, crystalline cellulose, dextrin, kaolin, calcium carbonate, and silicon dioxide. Examples of binders include polyvinyl alcohol, polyvinyl ether, ethyl cellulose, methyl cellulose, gum arabic, tragacanth, gelatin, shellac, hydroxypropyl cellulose, hydroxypropyl methyl cellulose, calcium citrate, dextrin, and pectin. Examples of lubricants include magnesium stearate, talc, polyethylene glycol, silica, and hardened vegetable oil. As coloring agents, any coloring agents that are approved to be added to pharmaceuticals can be used. As flavoring agents, cocoa powder, menthol, aromatic acids, peppermint oil, borneol, cinnamon powder, and the like can be used. Tablets and granules can be coated with sugar, gelatin, and other coatings as appropriate. In addition, preservatives, antioxidants, and the like may be added as necessary.

For producing liquid preparations for oral administration, such as emulsions, syrups, suspensions, or solutions, commonly used inert diluents such as water or vegetable oils can be used. Adjuvants such as moistening agents, suspension aids, sweeteners, aromatic agents, colorants, or preservatives can be added to liquid formulations. After a liquid formulation is prepared, capsules of such as gelatin may be filled with the liquid formulation.

Examples of pharmaceutical compositions for parenteral administration, such as solvents or suspensions used for producing injections, suppositories, or the like, include water, propylene glycol, polyethylene glycol, benzyl alcohol, ethyl oleate, and lecithin. Examples of bases used for producing suppositories include cocoa butter, emulsified cocoa butter, and lauric butter. The method of preparing preparations is not particularly limited, and any method commonly used in the industry can be used.

In a case of preparing a pharmaceutical composition in the form of an injection, as carriers, it is possible to use, for example: diluents such as water, ethyl alcohol, and propylene glycol; pH adjusters or buffer agents such as sodium citrate, sodium acetate, and sodium phosphate; and stabilizers such as ethylenediamine tetraacetic acid, thioglycolic acid, and thiolactic acid. A sufficient amount of salt, glucose, mannitol, glycerin, or the like may be contained in the composition to prepare an isotonic solution, and a dissolution assistant, a soothing agent, a local anesthetic, and the like may be added.

In a case of preparing a pharmaceutical composition in the form of ointment, such as a paste, cream, or a gel, commonly used bases, stabilizers, moistening agents, preservatives, or the like can be used as necessary and a pharmaceutical composition can be prepared by mixing components through a usual method. As bases, for example, white vaseline, polyethylene, paraffin, glycerin, cellulose derivatives, polyethylene glycol, silicone, or bentonite can be used. As preservatives, for example, methyl paraoxybenzoate, ethyl paraoxybenzoate, or propyl paraoxybenzoate can be used. In a case of preparing a pharmaceutical composition in the form of a patch, the above-described ointment, cream, gel, paste, or the like can be applied to the surface of a usual support according to a usual method. As supports, woven or non-woven fabrics made of cotton or synthetic fibers, films made of soft vinyl chloride, polyethylene, or polyurethanes, foam sheets, or the like can be suitably used.

The amount of active ingredient in the composition is not particularly limited as long as the composition can be used for a desired purpose, and the amount thereof can be appropriately increased or decreased depending on the age, weight, and sex of patients, the purpose of administration, the symptoms, and the like.

### (Differentiation Method)

According to a second aspect, there is provided a method of differentiating hepatocytes with bile ductular proliferation from hepatocytes without bile ductular proliferation, the method including a step of: detecting laminin γ2 single chain or a nucleic acid encoding the same in a specimen; in which in a case where laminin γ2 single chain or a nucleic acid encoding the same is detected, the specimen is determined to contain hepatocytes with bile ductular proliferation, and in a case where laminin γ2 single chain or a nucleic acid encoding the same is not detected, the specimen is determined not to contain hepatocytes with bile ductular proliferation.

The step of detecting laminin γ2 single chain or a nucleic acid encoding the same in a specimen can be performed through the above-described method.

The presence or absence of laminin γ2 single chain or a nucleic acid

encoding the same in a specimen can be used for various applications in addition to differentiation of hepatocytes with bile ductular proliferation from hepatocytes without bile ductular proliferation. Although not intended to be limiting, examples thereof include: 1) prognosis prediction of liver failure and diagnosis of the necessity or indications for liver transplantation (differentiation of necessity of liver transplantation); 2) prognosis prediction of patients with hepatic fibrosis (differentiation of necessity of liver transplantation); 3) diagnosis of biliary atresia; 4) prognosis prediction of primary biliary cholangitis and primary sclerosing cholangitis (differentiation of necessity of liver transplantation); 5) prediction of the effect of drug therapy for pruritus due to cholestasis (diagnosis of efficacy of Remitch); and 6) discrimination of liver regeneration.

For example, quantitative analysis of laminin γ2 single chain or a nucleic acid encoding the same in a specimen derived from a subject suspected of having a specific liver disease causing bile ductular proliferation, such as liver failure, can be performed to determine that, for example, the prognosis for liver failure is poor in a case where laminin γ2 single chain or a nucleic acid encoding the same is increased and the prognosis for liver failure is favorable in a case where laminin γ2 single chain or a nucleic acid encoding the same is decreased.

### (Biomarker)

According to a third aspect, there is provided a biomarker for detecting hepatocytes with bile ductular proliferation, containing: laminin γ2 single chain or a nucleic acid encoding the same.

A biomarker containing laminin γ2 single chain or a nucleic acid encoding the same can be used to detect hepatocytes with bile ductular proliferation and detect a liver disease causing bile ductular proliferation, such as primary biliary cholangitis, acute hepatitis, fulminant hepatitis, liver failure, primary sclerosing cholangitis, liver fibrosis, biliary atresia, and cholestasis.

### (Kit)

According to a fourth aspect, there is provided a kit for detecting hepatocytes with bile ductular proliferation, including: a reagent for detecting laminin γ2 single chain or a nucleic acid encoding the same.

Examples of reagents for detecting laminin γ2 single chain or a nucleic acid encoding the same include an anti-laminin γ2 single chain antibody. Such an antibody can be used in immunoassays that utilize antigen-antibody reactions with laminin γ2 single chain. The kit may include other reagents and devices necessary to measure the amount of laminin γ2 single chain. The kit may further include: a reagent for detecting CK19 or a nucleic acid encoding the same.

In a certain embodiment, a kit used in an immunoassay may include: a microtiter plate; an anti-laminin γ2 single chain antibody for capture; an anti-laminin γ2 single chain antibody labeled with alkaline phosphatase or peroxidase; and an alkaline phosphatase substrate (such as NPP) or peroxidase substrates (such as DAB, TMB, and OPD).

In such a kit, first, a capture antibody is immobilized on a microtiter plate, a specimen is appropriately diluted and added thereto and then incubated, and the sample is removed and washed. Next, a labeled antibody is added thereto and then incubated, and a substrate is added thereto to develop color. By measuring color development using a microtiter plate reader or the like, the amount of laminin γ2 single chain can be determined.

In another embodiment of the test kit, the kit may include: a microtiter plate; an anti-laminin γ2 single chain antibody for capture; an anti-laminin γ2 single chain antibody as a primary antibody; an anti-laminin γ2 single chain antibody labeled with alkaline phosphatase or peroxidase as a secondary antibody; and alkaline phosphatase (such as NPP) or peroxidase substrates (such as DAB, TMB, and OPD). The capture antibody and the primary antibody recognize different epitopes.

In such a kit, first, a capture antibody is immobilized on a microtiter plate, a specimen is appropriately diluted and added thereto and then incubated, and the sample is removed and washed. Subsequently, a primary antibody is added thereto, incubated, and washed, an enzyme-labeled secondary antibody is added thereto and incubated, and then a substrate is added thereto to develop color. By measuring color development using a microtiter plate reader or the like, the amount of laminin γ2 single chain can be determined. By using the secondary antibody, the reaction can be amplified and detection sensitivity can be increased.

It is also preferable that each kit further contain necessary buffers, enzyme reaction stop solutions, microplate readers, and the like.

Labeled antibodies are not limited to enzyme-labeled antibodies, and may be antibodies labeled with radioactive substances (such as 25I, 131I, 35S, and 3H), fluorescent substances (such as fluorescein isothiocyanate, rhodamine, dansyl chloride, phycoerythrin, tetramethylrhodamine isothiocyanate, and a near-infrared fluorescent material), luminescent substances (such as a luciferase, luciferin, and aequorin), and nanoparticles (such as gold colloids and quantum dots).

Alternatively, a biotinylated antibody can be used as the labeled antibody, and labeled avidin or streptavidin can be added to the kit.

Hereinafter, the present invention will be described more specifically with reference to examples, but the present invention is not limited thereto.

### EXAMPLES

### [Materials and Methods]

### Creation of Mouse Models Using Animals and NASH-Inducing Diet

Male 8-week-old C57BL6/J mice were purchased from NINOX. After two weeks of breeding, the mice were each fed a high-fat diet (HFD, D12492, Research Diets Inc.), an atherogenic plus high-fat diet (Ath+HFD, D06061403, Research Diets Inc.), and an ultra-high fat choline-deficient methionine-reduced diet (CDAA, A06071302, Research Diets Inc.). Liver tissues were removed after 2, 4, 8, and 10 weeks of feeding. The removed liver tissues were immobilized with 10% formalin.

### Immunohistochemistry (IHC)

After removing the mouse liver tissues, these were immobilized with 10% formaldehyde, paraffin blocks (FFPE) were prepared, and serial thin sections were prepared. The FFPE sections were deparaffinized and were reacted with 0.05% proteinase 24 (Sigma-Aldrich Co. LLC.: P8038) for 1 minute or with Antigen Activation Solution pH 9 (Nichirei Corporation: 415201) at 95° C. for 10 minutes for antigen activation. 3% H₂O₂ was used to cause a reaction at room temperature for 15 minutes to inhibit endogenous peroxidase activity. To suppress non-specific binding, Nichirei mouse stain kit (Nichirei Biosciences inc.: 414322) was used when using a monoclonal antibody. Those sections were incubated with anti-human laminin γ2 (Ln-γ2) rabbit lgG (10 mg/mL, S3, prepared by the present inventors), cytokeratin 19 (CK19) (1:100, ab133496, Abcam), a polyclonal antibody of anti-human laminin α3 (Ln-α3) rabbit IgG (10 mg/mL, bs-1969R, Bioss) and a monoclonal antibody of anti-human laminin β3 (Ln-β3) mouse lgG (20 mg/mL, 8A, donated by Professor Kaoru Miyazaki, Yokohama City University) at 4° C. overnight. SignalStain Antibody Diluent (Cell Signaling Technology: #8112) was used for diluting all antibodies. After washing the FFPE sections with PBS-T, Histone Fine Simple Stain Mouse MAX-PO(R) (Nichirei Biosciences inc.: 414341) or Histone Fine Simple Stain Mouse MAX-PO(M) (Nichirei Biosciences inc.: 414322) was used as secondary antibodies to cause a reaction at room temperature for 10 minutes. After washing the sections with PBS-T, a Simple Stain DAB solution (Nichirei Biosciences inc.: 415174) was used to cause a reaction for 1 minute and 30 seconds or 10 minutes, and each antigen was detected in dark brown. Hematoxylin was used for counterstaining. After dehydrating and sealing the sections, the stained tissue images were captured and observed on virtual slides using Aperio ScanScope CS2(Leica).

### [Results]

### 1. Confirmation of Reactivity of Each Laminin Chain Antibody Through IHC

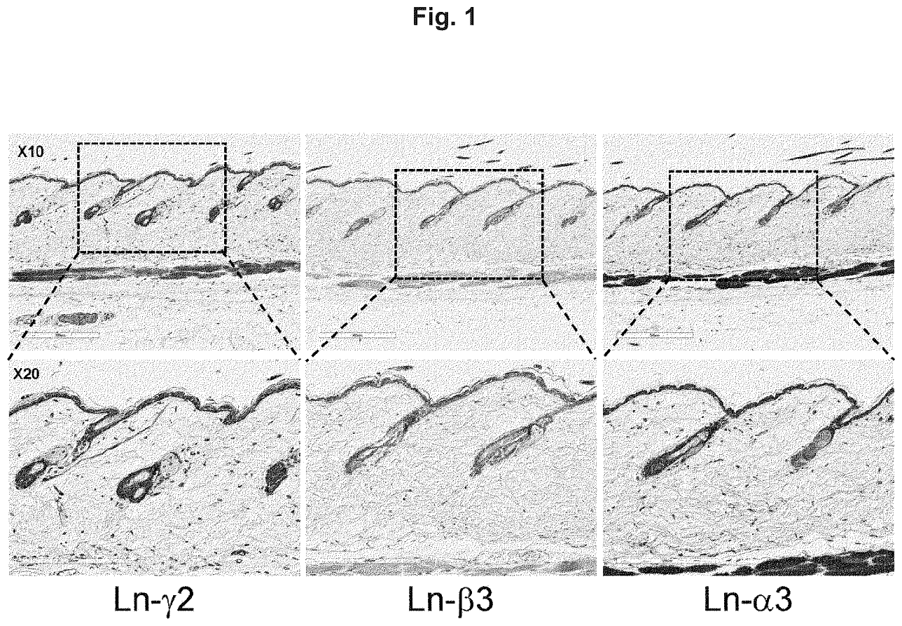

To clarify expression and localization of laminin-γ2 (Ln-γ2) chain in non-alcoholic steatohepatitis (NASH) livers, immunohistochemistry (IHC) using livers from two different strains of NASH mouse models was performed. First, to confirm immunoreactivity of human laminin chain antibodies for Ln-γ2, laminin-α3 (Ln-α3), and laminin-β3 (Ln-β3) to mouse laminin chains, IHC was performed using mouse skin FFPE sections. As a result, each laminin chain of laminin-332 (a heterotrimer composed of Ln-α3, β3, and γ2 chains) expressed in the basement membrane was detected, and the reactivity of each antibody was confirmed (FIG. 1).

### 2. Ultra-High Fat Choline-Deficient Methionine-Reduced Diet (CDAA)-Induced NASH Mouse Models

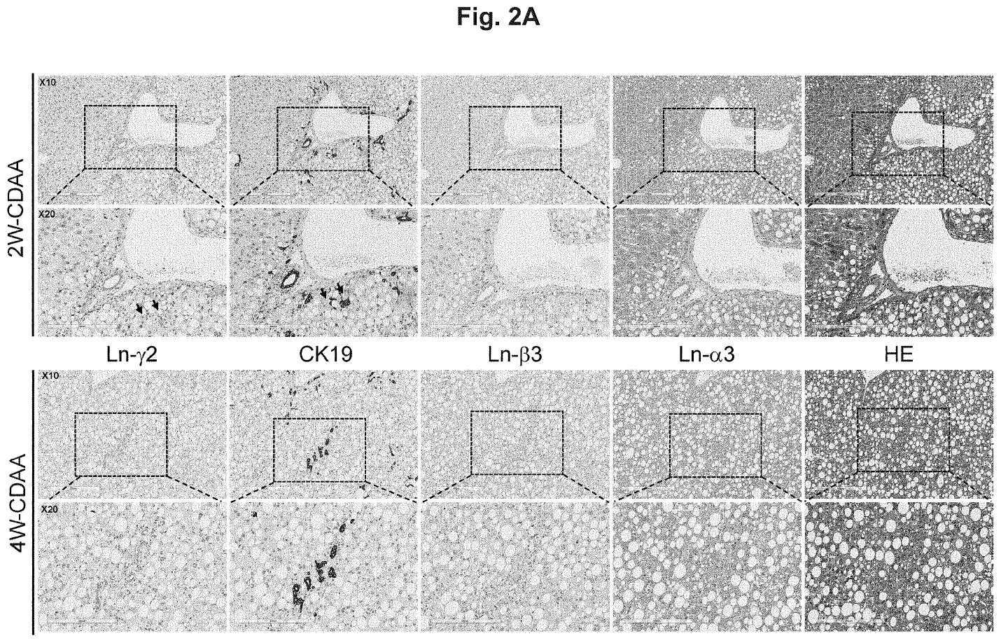

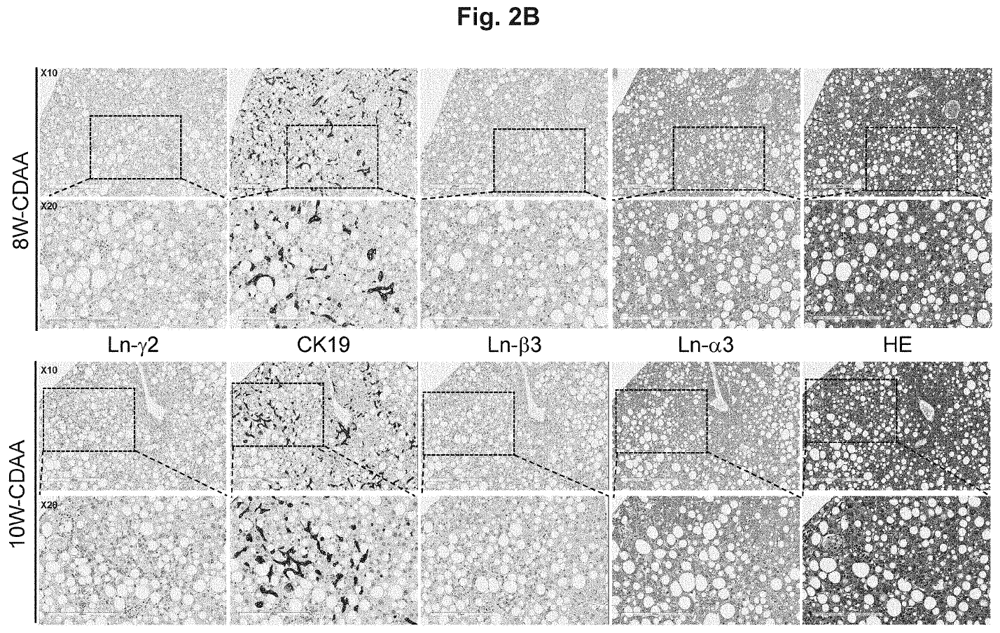

First, to create mouse models that aggressively develop NASH, mice were fed an ultra-high fat choline-deficient methionine-reduced diet (CDAA) for 2, 4, 8, and 10 weeks starting at 2 weeks of age. FFPE sections of livers removed from mice euthanized at each period were prepared, and expression and localization of Ln-γ2 chains, cytokeratin 19 (CK19), Ln-β3 chains, and Ln-α3 chains in the liver tissues were examined through IHC. Ectopic fat accumulation was observed in the mouse liver after 2 weeks of CDAA feeding, suggesting that fatty liver had developed (FIG. 2A, upper panel). In addition, in the livers after 2 and 4 weeks of CDAA feeding, CK19-positive bile duct epithelial-like cells were observed in and around the bile ducts near the portal vein, and Ln-γ2 chains were weakly co-expressed (FIG. 2A, upper panel, arrows). Furthermore, in the liver tissues after 8 and 10 weeks of CDAA feeding, numerous CK19-positive bile ductules proliferated, and it was found that Ln-γ2 chains co-localized with these bile duct epithelial-like cells (FIG. 2B). In addition, although expression of the Ln-α3 chains was observed in some bile duct epithelial-like cells, expression of the Ln-β3 chains was not observed, suggesting that the Ln-α3 chains may be associated with other chains of laminin, and Ln-γ2 chains are expressed as single chains because they do not associate with any chains other than the Ln-α3 and Ln-β3 chains.

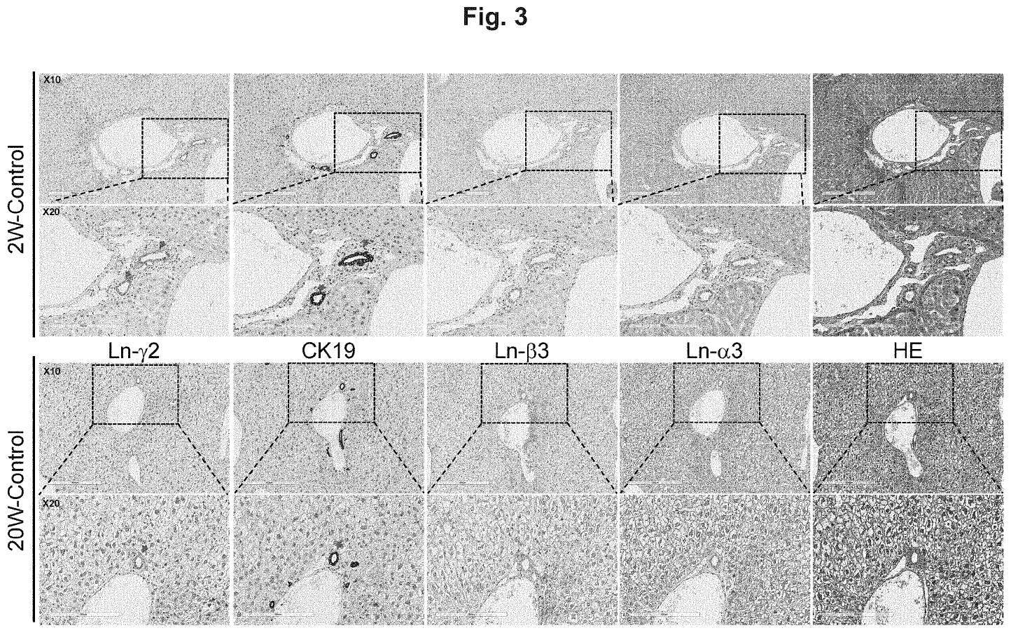

On the other hand, mouse livers after 2 and 20 weeks of feeding a normal diet (ND) (control livers) were used to examine expression of each molecule in the control livers through IHC. In the liver after 2 weeks of ND feeding, weak expression of Ln-γ2 chains was detected in CK19-positive bile duct epithelial-like cells near the portal vein, but expression of laminin α3 and β3 chains could not be detected (FIG. 3, upper panel, arrowhead). Furthermore, expression of Ln-γ2 single chain in control liver showed a decreasing trend at 20 weeks of age (FIG. 3, lower panel, arrowhead). As described above, it was found that the Ln-γ2 chain was expressed in the bile duct epithelial cells of the control liver in a single chain state. Furthermore, its expression decreases as liver tissue matures, suggesting that Ln-γ2 single chain may play some role in liver development.

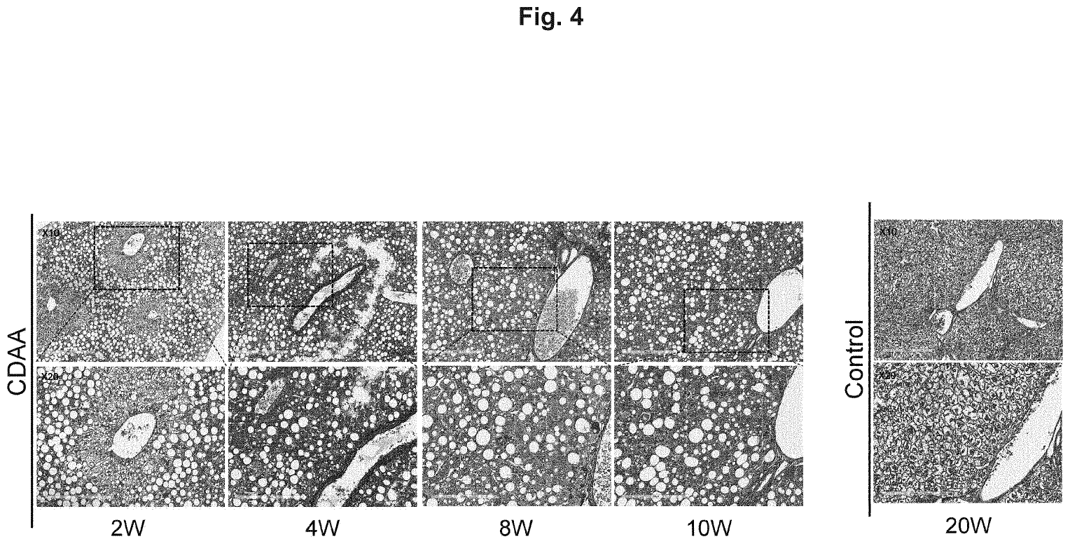

Furthermore, in order to examine fibrotic states of the livers of mice that had been fed CDAA for 2, 4, 8, and 10 weeks, an examination was performed through Azan staining. The results are shown in FIG. 4. Aniline blue-positive collagen fiber proteins accumulated around the central vein of the livers of all mice. These are thought to be collagen fiber proteins such as elastin and collagen that form blood vessel walls. On the other hand, in the livers and after 2 and 4 weeks of CDAA feeding, lipid droplets were observed, but no accumulation of collagen fiber proteins could be detected. Furthermore, in the livers after 8 and 10 weeks of CDAA feeding, accumulation of collagen fiber proteins such as collagen in hepatocytes was observed, suggesting that liver fibrosis was progressing. On the other hand, in a control mouse (20 weeks), collagen fiber proteins were expressed around the portal vein, but no significant accumulation of collagen fiber proteins was observed in the liver tissue.

### 3. Atherogenic Plus High-Fat (Ath+HF) Diet-Induced NASH Mouse Models

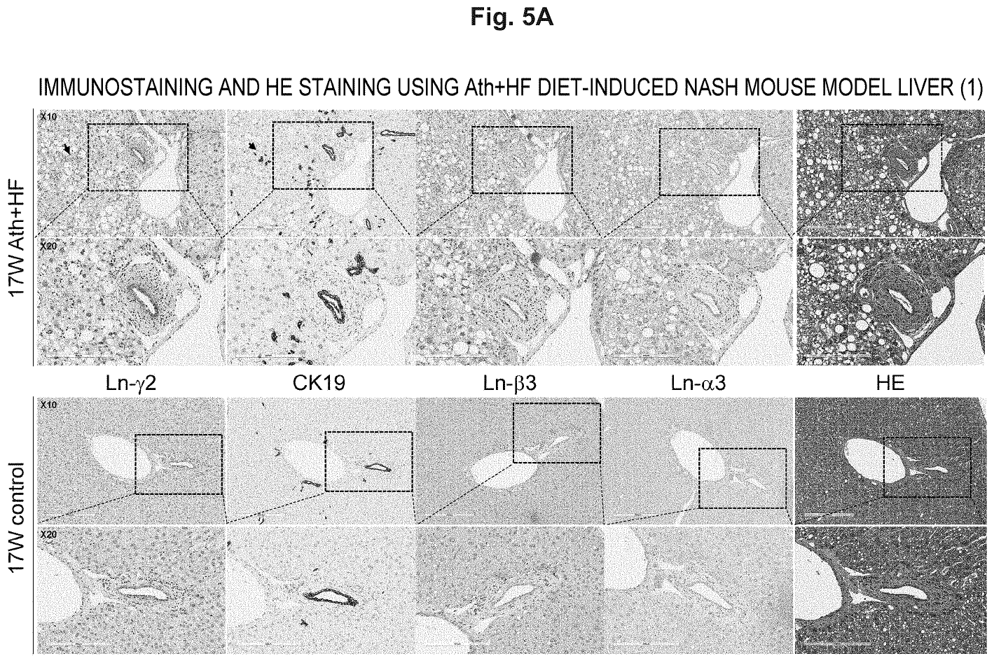

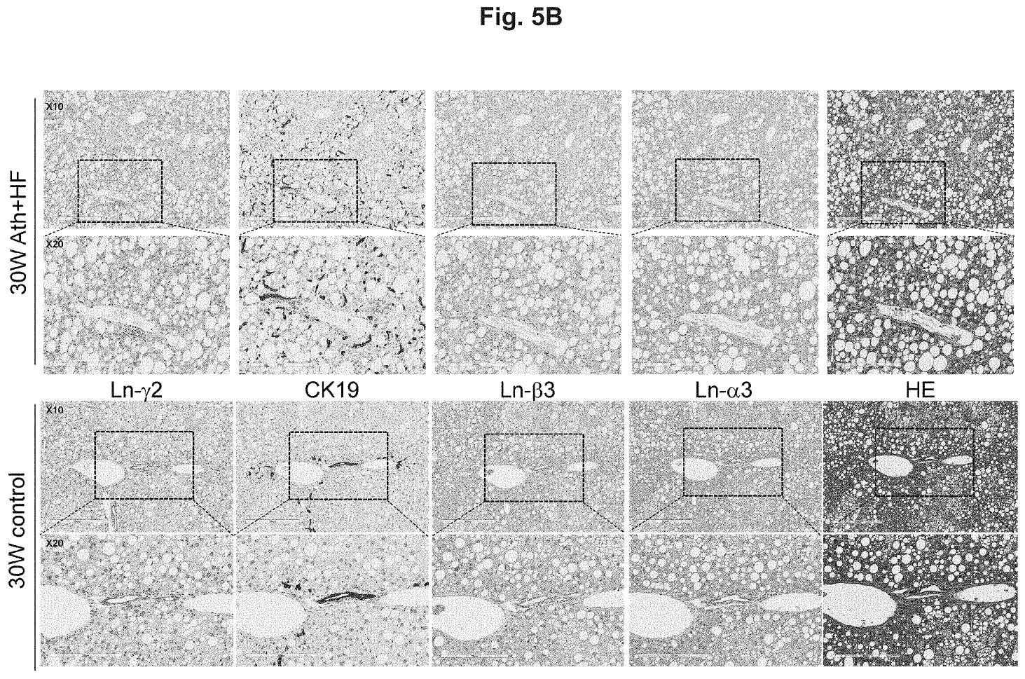

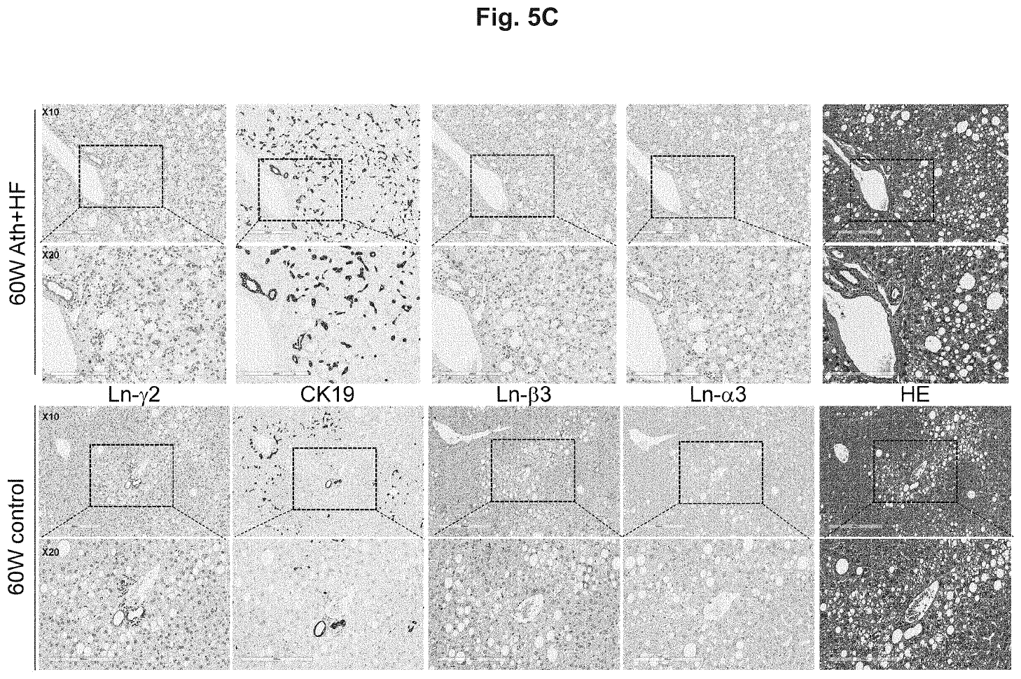

Next, to verify reproducibility of the results obtained with the above-described NASH mouse models, an examination was performed using atherogenic plus high-fat (Ath+HF) diet-induced NASH mouse model livers. The results are shown in FIGS. 5A to 5C. Since the onset of NASH occurs more slowly in these models than in the CDAA-induced models, the expression and localization of Ln-γ2, CK19, Ln-B3 chains, and Ln-α3 chains were examined through IHC using livers after 17, 30, and 60 weeks of Ath+HF feeding.

As a result, ectopic fat accumulation in hepatocytes was observed after 17 weeks of Ath+HF feeding. In addition, expression of CK19 and Ln-γ2 chains was detected in bile duct epithelial cells around the portal vein in both control and NASH livers. Furthermore, in the NASH liver, CK19-positive immature bile ductular proliferation was frequently detected in areas away from the portal vein, whereas no bile ductular proliferation was observed in the control liver. At this time, some CK19-positive bile ductular epithelial cells express very weak Ln-γ2 chains, but no expression of Ln-β3 chains is detected (FIG. 5A, upper panel, arrow).

After 30 and 60 weeks of Ath+HF feeding, it was found that expression of Ln-γ2 chains and CK19 increased with age in weeks (FIGS. 5B and 5C). In particular, after 60 weeks of Ath+HF feeding, almost all CK19-positive bile ductular epithelial cells and bile duct epithelial cells were shown to express Ln-γ2 single chain (FIG. 5C). Expression of Ln-γ2 chains and CK19 was observed in the bile ductular epithelial cells and the bile ducts near the portal vein, but no expression of Ln-β3 chains was observed (FIGS. 5B and 5C).

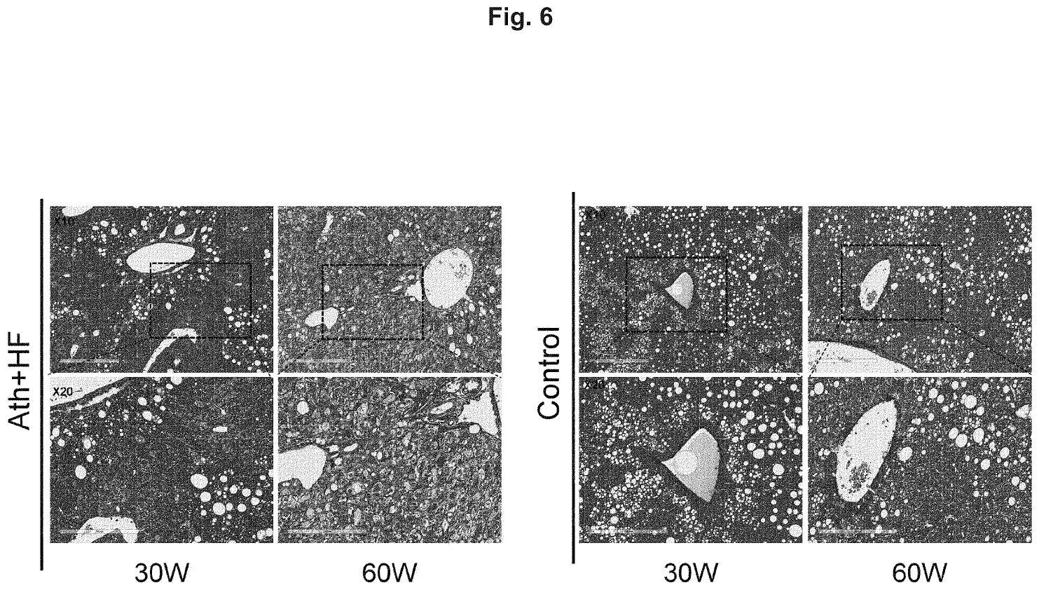

Next, the fibrotic states of the livers of the mice that had been fed Ath+HF were examined through Azan staining. As a result, collagen fiber proteins were observed around the portal vein in the liver after 30 weeks of Ath+HF feeding, and weak collagen fiber protein accumulation was observed in the liver tissue. Furthermore, after 60 weeks of Ath+HF feeding, the accumulation of collagen fiber proteins in the liver tissue significantly increased compared to after 30 weeks of Ath+HF feeding, indicating that liver fibrosis was progressing (FIG. 6). On the other hand, when control livers were similarly subjected to Azan staining, collagen fiber proteins were detected around the central vein and portal vein, but no accumulation thereof was observed in the liver tissue (FIG. 6).

Immunohistochemistry using these two NASH models revealed that as the pathology of mouse NASH progresses, CK19-positive bile ductular proliferation occurs, and Ln-γ2 chains are co-expressed as single chains in bile ductular epithelial cells.

In addition, weak expression of Ln-γ2 single chain was observed in bile duct epithelial cells in the livers of young mice, but the expression thereof was observed to be decreased with aging. This is the first report of Ln-γ2 single chain expression from normal tissue. This suggests that Ln-γ2 single chain may affect the development, differentiation and the like of mouse liver.

1. A method of detecting a biomarker for detecting hepatocytes with bile ductular proliferation which comprises the step of:
detecting laminin γ2 single chain or a nucleic acid encoding the same as the biomarker in a specimen.

2. The method according to claim 1, wherein
hepatocytes with bile ductular proliferation are cells having a liver disease causing bile ductular proliferation.

3. The method according to claim 2, wherein
the liver disease causing bile ductular proliferation is selected from the group consisting of primary biliary cholangitis, acute hepatitis, fulminant hepatitis, liver failure, primary sclerosing cholangitis, hepatic fibrosis, biliary atresia, and cholestasis.

4. The method according to claim 1, wherein in a case where laminin γ2 single chain or a nucleic acid encoding the same is detected, the specimen is likely to contain hepatocytes with bile ductular proliferation or hepatocytes having a liver disease causing bile ductular proliferation.

5. The method according to claim 1, which further comprises the step of:
detecting a bile ductular proliferation-related marker other than laminin γ2 single chain or a nucleic acid encoding the same.

6. The method according to claim 5, wherein
the bile ductular proliferation-related marker is one or a plurality of makers selected from the group consisting of SOX9, AQP1, CK7, EpCAM, and Dlk1.

7. The method according to claim 1, wherein the detection of laminin γ2 single chain or a nucleic acid encoding the same is performed over time.

8. The method according to claim 7, wherein
change in the amount of laminin γ2 single chain in a specimen or a nucleic acid encoding the same over time is compared with change in the amount of CK19 in the specimen or a nucleic acid encoding the same over time.

9. The method according to claim 1, wherein
the specimen is derived from a subject suspected of having hepatocytes with bile ductular proliferation or hepatocytes having a liver disease causing bile ductular proliferation.

10. The method according to claim 9, wherein
the specimen is derived from blood or urine of the subject.

11. A method of differentiating hepatocytes with bile ductular proliferation from hepatocytes without bile ductular proliferation which comprises the step of:
detecting laminin γ2 single chain or a nucleic acid encoding the same in a specimen, wherein
in a case where laminin γ2 single chain or a nucleic acid encoding the same is detected, the specimen is determined to contain hepatocytes with bile ductular proliferation, and
in a case where laminin γ2 single chain or a nucleic acid encoding the same is not detected, the specimen is determined not to contain hepatocytes with bile ductular proliferation.

12. The method according to claim 11, wherein
hepatocytes with bile ductular proliferation are cells having a liver disease causing bile ductular proliferation.

13. The method according to claim 12, wherein
the liver disease causing bile ductular proliferation is selected from the group consisting of primary biliary cholangitis, acute hepatitis, fulminant hepatitis, liver failure, primary sclerosing cholangitis, hepatic fibrosis, biliary atresia, and cholestasis.

14. A biomarker for detecting hepatocytes with bile ductular proliferation, comprising:
laminin γ2 single chain or a nucleic acid encoding the same.

15. The biomarker according to claim 14, wherein
hepatocytes with bile ductular proliferation are cells having a liver disease causing bile ductular proliferation.

16. A kit for detecting hepatocytes with bile ductular proliferation, comprising:
a reagent for detecting laminin γ2 single chain or a nucleic acid encoding the same.

17. The kit according to claim 16, wherein
hepatocytes with bile ductular proliferation are cells having a liver disease causing bile ductular proliferation.

18. The kit according to claim 17, wherein
the liver disease causing bile ductular proliferation is selected from the group consisting of primary biliary cholangitis, acute hepatitis, fulminant hepatitis, liver failure, primary sclerosing cholangitis, hepatic fibrosis, biliary atresia, and cholestasis.

19. The kit according to any claim 16, further comprising:
a reagent for detecting CK19 or a nucleic acid encoding the same.


SEQUENCE LISTING (2 sequences)

SEQ ID NO: 1 - protein, 1193 aa, Homo sapiens
MPALWLGCCLCFSLLLPAARATSRREVCDCNGKSRQCIFDRELHRQTGNGFRCLNCNDNTDGIHCEKCKNGFYRHRERDRCLPCNCNSKGSLSARCDNSGRCSCKPGVTGARCDRCLPGFHMLTDAGCTQDQRLLDSKCDCDPAGIAGPCDAGRCVCKPAVTGERCDRCRSGYYNLDGGNPEGCTQCFCYGHSASCRSSAEYSVHKITSTFHQDVDGWKAVQRNGSPAKLQWSQRHQDVFSSAQRLDPVYFVAPAKFLGNQQVSYGQSLSFDYRVDRGGRHPSAHDVILEGAGLRITAPLMPLGKTLPCGLTKTYTFRLNEHPSNNWSPQLSYFEYRRLLRNLTALRIRATYGEYSTGYIDNVTLISARPVSGAPAPWVEQCICPVGYKGQFCQDCASGYKRDSARLGPFGTCIPCNCQGGGACDPDTGDCYSGDENPDIECADCPIGFYNDPHDPRSCKPCPCHNGFSCSVMPETEEVVCNNCPPGVTGARCELCADGYFGDPFGEHGPVRPCQPCQCNNNVDPSASGNCDRLTGRCLKCIHNTAGIYCDQCKAGYFGDPLAPNPADKCRACNCNPMGSEPVGCRSDGTCVCKPGFGGPNCEHGAFSCPACYNQVKIQMDQFMQQLQRMEALISKAQGGDGVVPDTELEGRMQQAEQALQDILRDAQISEGASRSLGLQLAKVRSQENSYQSRLDDLKMTVERVRALGSQYQNRVRDTHRLITQMQLSLAESEASLGNTNIPASDHYVGPNGFKSLAQEATRLAESHVESASNMEQLTRETEDYSKQALSLVRKALHEGVGSGSGSPDGAVVQGLVEKLEKTKSLAQQLTREATQAEIEADRSYQHSLRLLDSVSRLQGVSDQSFQVEEAKRIKQKADSLSSLVTRHMDEFKRTQKNLGNWKEEAQQLLQNGKSGREKSDQLLSRANLAKSRAQEALSMGNATFYEVESILKNLREFDLQVDNRKAEAEEAMKRLSYISQKVSDASDKTQQAERALGSAAADAQRAKNGAGEALEISSEIEQEIGSLNLEANVTADGALAMEKGLASLKSEMREVEGELERKELEFDTNMDAVQMVITEAQKVDTRAKNAGVTIQDTLNTLDGLLHLMDQPLSVDEEGLVLLEQKLSRAKTQINSQLRPMMSELEERARQQRGHLHLLETSIDGILADVKNLENIRDNLPPGCYNTQALEQQ

SEQ ID NO: 2 - DNA, 5398 bp, Homo sapiens
  source 1..5398: mol_type = genomic DNA
acagcggagcgcagagtgagaaccaccaaccgaggcgccgggcagcgacccctgcagcggagacagagactgagcggcccggccccgccatgcctgcgctctggctgggctgctgcctctgcttctcgctcctcctgcccgcagcccgggccacctccaggagggaagtctgtgattgcaatgggaagtccaggcagtgtatctttgatcgggaacttcacagacaaactggtaatggattccgctgcctcaactgcaatgacaacactgatggcattcactgcgagaagtgcaagaatggcttttaccggcacagagaaagggaccgctgtttgccctgcaattgtaactccaaaggttctcttagtgctcgatgtgacaactccggacggtgcagctgtaaaccaggtgtgacaggagccagatgcgaccgatgtctgccaggcttccacatgctcacggatgcggggtgcacccaagaccagagactgctagactccaagtgtgactgtgacccagctggcatcgcagggccctgtgacgcgggccgctgtgtctgcaagccagctgtcactggagaacgctgtgataggtgtcgatcaggttactataatctggatggggggaaccctgagggctgtacccagtgtttctgctatgggcattcagccagctgccgcagctctgcagaatacagtgtccataagatcacctctacctttcatcaagatgttgatggctggaaggctgtccaacgaaatgggtctcctgcaaagctccaatggtcacagcgccatcaagatgtgtttagctcagcccaacgactagaccctgtctattttgtggctcctgccaaatttcttgggaatcaacaggtgagctatggtcaaagcctgtcctttgactaccgtgtggacagaggaggcagacacccatctgcccatgatgtgattctggaaggtgctggtctacggatcacagctcccttgatgccacttggcaagacactgccttgtgggctcaccaagacttacacattcaggttaaatgagcatccaagcaataattggagcccccagctgagttactttgagtatcgaaggttactgcggaatctcacagccctccgcatccgagctacatatggagaatacagtactgggtacattgacaatgtgaccctgatttcagcccgccctgtctctggagccccagcaccctgggttgaacagtgtatatgtcctgttgggtacaaggggcaattctgccaggattgtgcttctggctacaagagagattcagcgagactggggccttttggcacctgtattccttgtaactgtcaagggggaggggcctgtgatccagacacaggagattgttattcaggggatgagaatcctgacattgagtgtgctgactgcccaattggtttctacaacgatccgcacgacccccgcagctgcaagccatgtccctgtcataacgggttcagctgctcagtgatgccggagacggaggaggtggtgtgcaataactgccctcccggggtcaccggtgcccgctgtgagctctgtgctgatggctactttggggacccctttggtgaacatggcccagtgaggccttgtcagccctgtcaatgcaacaacaatgtggaccccagtgcctctgggaattgtgaccggctgacaggcaggtgtttgaagtgtatccacaacacagccggcatctactgcgaccagtgcaaagcaggctacttcggggacccattggctcccaacccagcagacaagtgtcgagcttgcaactgtaaccccatgggctcagagcctgtaggatgtcgaagtgatggcacctgtgtttgcaagccaggatttggtggccccaactgtgagcatggagcattcagctgtccagcttgctataatcaagtgaagattcagatggatcagtttatgcagcagcttcagagaatggaggccctgatttcaaaggctcagggtggtgatggagtagtacctgatacagagctggaaggcaggatgcagcaggctgagcaggcccttcaggacattctgagagatgcccagatttcagaaggtgctagcagatcccttggtctccagttggccaaggtgaggagccaagagaacagctaccagagccgcctggatgacctcaagatgactgtggaaagagttcgggctctgggaagtcagtaccagaaccgagttcgggatactcacaggctcatcactcagatgcagctgagcctggcagaaagtgaagcttccttgggaaacactaacattcctgcctcagaccactacgtggggccaaatggctttaaaagtctggctcaggaggccacaagattagcagaaagccacgttgagtcagccagtaacatggagcaactgacaagggaaactgaggactattccaaacaagccctctcactggtgcgcaaggccctgcatgaaggagtcggaagcggaagcggtagcccggacggtgctgtggtgcaagggcttgtggaaaaattggagaaaaccaagtccctggcccagcagttgacaagggaggccactcaagcggaaattgaagcagataggtcttatcagcacagtctccgcctcctggattcagtgtctcggcttcagggagtcagtgatcagtcctttcaggtggaagaagcaaagaggatcaaacaaaaagcggattcactctcaagcctggtaaccaggcatatggatgagttcaagcgtacacagaagaatctgggaaactggaaagaagaagcacagcagctcttacagaatggaaaaagtgggagagagaaatcagatcagctgctttcccgtgccaatcttgctaaaagcagagcacaagaagcactgagtatgggcaatgccactttttatgaagttgagagcatccttaaaaacctcagagagtttgacctgcaggtggacaacagaaaagcagaagctgaagaagccatgaagagactctcctacatcagccagaaggtttcagatgccagtgacaagacccagcaagcagaaagagccctggggagcgctgctgctgatgcacagagggcaaagaatggggccggggaggccctggaaatctccagtgagattgaacaggagattgggagtctgaacttggaagccaatgtgacagcagatggagccttggccatggaaaagggactggcctctctgaagagtgagatgagggaagtggaaggagagctggaaaggaaggagctggagtttgacacgaatatggatgcagtacagatggtgattacagaagcccagaaggttgataccagagccaagaacgctggggttacaatccaagacacactcaacacattagacggcctcctgcatctgatggaccagcctctcagtgtagatgaagaggggctggtcttactggagcagaagctttcccgagccaagacccagatcaacagccaactgcggcccatgatgtcagagctggaagagagggcacgtcagcagaggggccacctccatttgctggagacaagcatagatgggattctggctgatgtgaagaacttggagaacattagggacaacctgcccccaggctgctacaatacccaggctcttgagcaacagtgaagctgccataaatatttctcaactgaggttcttgggatacagatctcagggctcgggagccatgtcatgtgagtgggtgggatggggacatttgaacatgtttaatgggtatgctcaggtcaactgacctgaccccattcctgatcccatggccaggtggttgtcttattgcaccatactccttgcttcctgatgctgggcaatgaggcagatagcactgggtgtgagaatgatcaaggatctggaccccaaagaatagactggatggaaagacaaactgcacaggcagatgtttgcctcataatagtcgtaagtggagtcctggaatttggacaagtgctgttgggatatagtcaacttattctttgagtaatgtgactaaaggaaaaaactttgactttgcccaggcatgaaattcttcctaatgtcagaacagagtgcaacccagtcacactgtggccagtaaaatactattgcctcatattgtcctctgcaagcttcttgctgatcagagttcctcctacttacaacccagggtgtgaacatgttctccattttcaagctggaagaagtgagcagtgttggagtgaggacctgtaaggcaggcccattcagagctatggtgcttgctggtgcctgccaccttcaagttctggacctgggcatgacatcctttcttttaatgatgccatggcaacttagagattgcatttttattaaagcatttcctaccagcaaagcaaatgttgggaaagtatttactttttcggtttcaaagtgatagaaaagtgtggcttgggcattgaaagaggtaaaattctctagatttattagtcctaattcaatcctacttttcgaacaccaaaaatgatgcgcatcaatgtattttatcttattttctcaatctcctctctctttcctccacccataataagagaatgttcctactcacacttcagctgggtcacatccatccctccattcatccttccatccatctttccatccattacctccatccatccttccaacatatatttattgagtacctactgtgtgccaggggctggtgggacagtggtgacatagtctctgccctcatagagttgattgtctagtgaggaagacaagcatttttaaaaaataaatttaaacttacaaactttgtttgtcacaagtggtgtttattgcaataaccgcttggtttgcaacctctttgctcaacagaacatatgttgcaagaccctcccatgggggcacttgagttttggcaaggctgacagagctctgggttgtgcacatttctttgcattccagctgtcactctgtgcctttctacaactgattgcaacagactgttgagttatgataacaccagtgggaattgctggaggaaccagaggcacttccaccttggctgggaagactatggtgctgccttgcttctgtatttccttggattttcctgaaagtgtttttaaataaagaacaattgttagatgcctggggtcagtttctgatttactaaactctaagcctatgatggtaccaaagtaatggaaatgaaaatgccacagaaatggcaataggtcctagagttagcatcttcagttcccacttctgagcatcttacttcgcccattccaaccatacaggattgatgatgctgtcaatgagatttttatgaggtttaactgatgctgtcttctaagcataaagctttattt